(19) United States
(12) Patent Application Publication
(10) Pub. No.: US 2024/0156583 A1
(43) Pub. Date: May 16, 2024

(54) FLEXIBLE COMPOSITE LAMINATE WITH HIGH SUTURE RETENTION STRENGTH AND METHOD OF MAKING SAME

(71) Applicant: International Polymer Engineering, Inc., Tempe, AZ (US)

(72) Inventors: Craig FITZPATRICK, Tempe, AZ (US); Anthony GREEN, Tempe, AZ (US); Kirk MYERS, Tempe, AZ (US)

(21) Appl. No.: 18/281,763

(22) PCT Filed: Mar. 14, 2022
(86) PCT No.: PCT/US2022/020273

§ 371 Date: Sep. 12, 2023

Related U.S. Application Data
(60) Provisional application No. 63/160,628, filed on Mar. 12, 2021.

(51) Int. Cl. A61F 2/00; B29C 70/34; B29C 70/68; B29K 27/18; B32B 5/02; B32B 5/12; B32B 5/26; B32B 27/12; B32B 27/32

(52) U.S. Cl. CPC A61F 2/0063; B29C 70/342; B29C 70/685; B29C 70/688; B32B 5/024; B32B 5/12; B32B 5/263; B32B 27/12; B32B 27/322; A61F 2002/0068; A61F 2210/0076; A61F 2220/0008; A61F 2240/001; B29K 2027/18; B29K 2995/0039; B29K 2995/0053; B29K 2995/0056; B29K 2995/0081; B32B 2250/40; B32B 2262/0253; B32B 2307/518; B32B 2307/54; B32B 2307/5825; B32B 2307/702; B32B 2535/00

(57) ABSTRACT

In a first aspect, the disclosure provides; a composite laminate. The laminate is made of: a first exterior layer comprising a biocompatible material; a second exterior layer comprising a biocompatible material; and a first inner layer comprising biocompatible threads running parallel to each other and oriented at zero degrees. The layers are laminated together. The disclosure further provides; a method for creating a biocompatible composite laminate. The method includes laying biocompatible threads parallel to one another to create a first middle thread layer on a first biocompatible material exterior layer, and placing a second biocompatible exterior material layer over the parallel biocompatible threads. The laminate is heated and compressed to bond the layers together.

### CROSS-REFERENCE TO RELATED APPLICATIONS

This application is the U.S. National Stage of International Application No. PCT/US2022/020273 filed on Mar. 14, 2022, which claims the benefit of priority from U.S. Prov. Pat. App. Ser. No. 63/160,628, filed under the same title on Mar. 12, 2021, the contents of which are fully incorporated herein by reference in their entireties.

### FIELD OF THE INVENTION

This invention relates to a flexible composite laminate with high tensile strength and suture retention strength for use in various medical repair procedures, organ support procedures, facial reconstruction procedures, pericardial/hernia patching procedures, and similar applications.

### BACKGROUND OF THE INVENTION

Expanded PTFE has been applied in the medical market for various long term implants like vascular grafts, hernia patches, suture, pericardial patches, facial reconstruction, etc. The unique non-woven micro-structure of expanded PTFE created by a thermal expansion of extruded and fibrillated PTFE resin has allowed for controlled tissue interactions. Those controlled tissue interactions have proven to facilitate either ingrowth or barrier type devices that support, replace, or repair existing tissues within the body. The high lubricity of PTFE, high flexibility, the biocompatibility of the final product, and long history of implantation has made this material a prime implant candidate.

However, one of the issues associated with the medical implant application of expanded PTFE is the ability to use the material in surgeries where the suture retention and prevent elongation may occur.

### SUMMARY OF THE INVENTION

In a first aspect, the disclosure provides; A composite laminate. The laminate is made of: a first exterior layer comprising a biocompatible material; a second exterior layer comprising a biocompatible material; and a first inner layer comprising biocompatible threads running parallel to each other and oriented at zero degrees. The layers are laminated together.

In a second aspect, the disclosure provides; A method for creating a biocompatible composite laminate. The method includes laying biocompatible threads parallel to one another to create a first middle thread layer on a first biocompatible material exterior layer, and placing a second biocompatible exterior material layer over the parallel biocompatible threads. The laminate is heated and compressed to bond the layers together.

### BRIEF DESCRIPTION OF THE DRAWINGS

The invention will be better understood from a reading of the following detailed description taken in conjunction with the drawings in which like reference designators are used to designate like elements, and in which:

FIG. 1A illustrates a composite laminate with three layers;

FIG. 1B illustrates a weaving rack;

FIG. 1C illustrates a laminating press;

FIGS. 2A through 2G illustrate different woven patterns;

FIG. 3A through 3C illustrate different knitted patterns; and

FIG. 4A through 4D illustrate different braided patterns.

### DETAILED DESCRIPTION OF THE PREFERRED EMBODIMENTS

This invention is described in preferred embodiments in the following description with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

### Definitions

As used herein “PTFE” is meant to refer to Polytetrafluorethylene, which is a synthetic fluoropolymer of tetrafluoroethylene.

As used herein “ePTFE” is meant to refer to PTFE that has undergone an expansion process. Generally, the expansion process begins with pure PTFE fine powder resin, which can form a paste by adding a lubricating agent. The paste is then extruded, which can be in rod or sheet form. The extrudate is heated while being secured in a device that will stretch it mechanically. The stretched part is heated to a temperature above 330° C. while being held in a device to prevent shrinkage. Expansion can be done along a single axis or biaxial along two axes. This sheet is heated and expanded. During the expansion process the density of the ePTFE can be set to specific densities, affecting properties of the ePTFE such as porosity. For example, there is an inverse relation between density and pore size: as the density of the ePTFE decreases, the porosity increases. Lower densities also correspond to higher air permeability, greater ability to flex, more compressibility, and greater tissue in-growth into implanted products. A higher density ePTFE corresponds to lower air permeability, lower ability to flex, stiffer, less compressible, and lower tissue in-growth.

As used herein “single-axis ePTFE” is meant to refer to ePTFE that has been stretched along a single axis. The ePTFE gains tensile strength and resists elongation along the axis of expansion.

As used herein “biaxial ePTFE” is meant to refer to ePTFE that has been stretched along two axes. Typically the two axes are perpendicular to each other.

As used herein “anisotropic” is meant to refer to a material that has a physical property that has a different value when measured in different directions. A common example is wood, which is stronger along the grain than across it.

As used herein “sintering” is meant to refer to applying heat and optionally also compression to a powdered material to lock it in an amorphous state. Often the heating of the material is accompanied by compression of the material.

As used herein “inter-nodal distance (IND)” is meant to refer to a measurement of the fibril length relative to the ratio of mechanical expansion. The microstructure of ePTFE can be described as roughly parallel-running groups of nodes with perpendicular fibers connecting them. The fibers connecting the nodes are called fibrils. It is the fibril length that is commonly used to determine the porosity in ePTFE materials; correspondingly, a larger IND equates to higher porosity, and a smaller IND equates to lower porosity.

As used herein “porosity” is meant to refer to the amount and size of pores in an ePTFE membrane. Porosity can be quantified by the internodal distance of the ePTFE membrane, or the density of the membrane. Non-Porous PTFE is not porous and does not have any pores, its density is 2.15 g/cc. High density/low porosity ePTFE has an internodal distance of <15 microns (0.015 mm) and a density of >0.85 g/cc. Medium density/medium porosity ePTFE has an internodal distance of 20-40 microns (0.020-0.040 mm) and a density of 0.40-0.60 g/cc. Low density/high porosity ePTFE has an internodal distance of 40-80 microns (0.040-0.080 mm) and a density of <0.35 g/cc.

As used herein “lamination adherence” is meant to refer to the bond created when layers are laminated together. Lamination creates a grip strength between the layers so that they grip adjacent layers and do not slide or shift on one another.

As used herein “woven” is not limited to the process of interlacing two yarns/threads so that they cross each other at right angles to produce a woven layer. “Woven” is used to include the result of knitting and braiding. Knitting is a method of constructing layer by interlocking a series of loops of one or more yarns/threads and a braid is a complex structure of pattern formed by intertwining three or more yarns/threads.

This disclosure presents a multi-layer composite laminate with high suture retention forces, low elongation (elongation measures the percentage change in length before fracture), and high tensile strengths in all axes of each layer, and high flexibility for applications in various tissue repair and organ support surgeries.

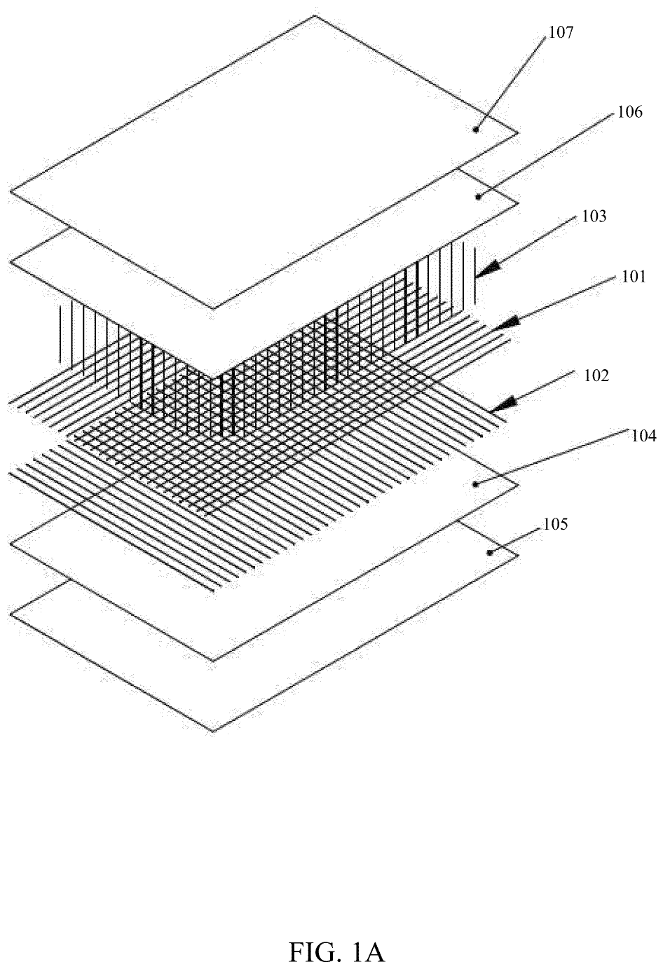

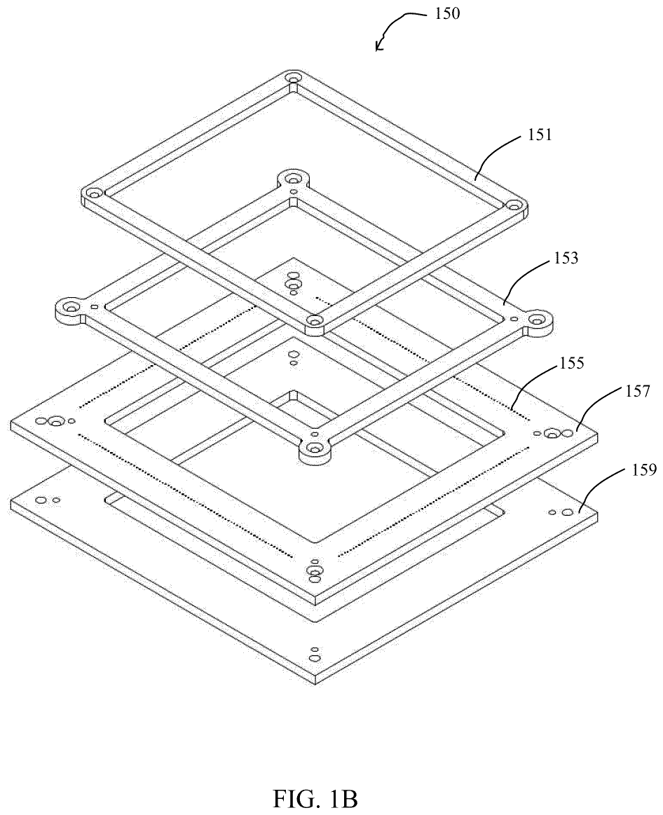

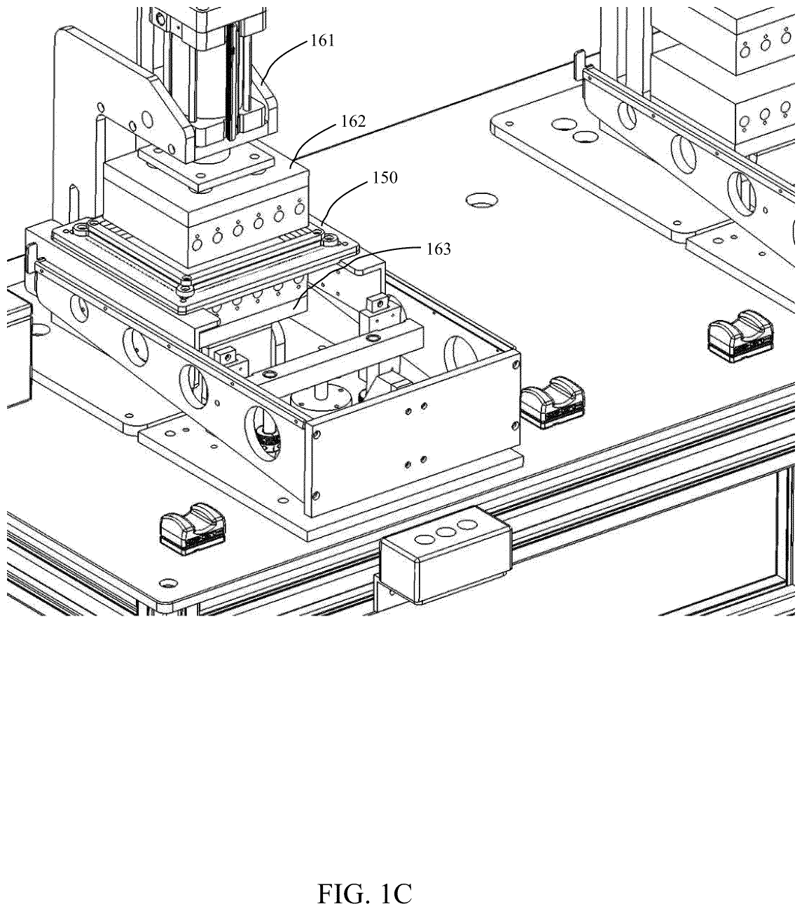

Referring to FIG. 1A, a multi-layer composite laminate 100 comprises a biocompatible membrane exterior layer 105, layers 101, 102, and 103 are tear resistant layers, layers 104 and 106 are amorphic binding layers, and another biocompatible membrane exterior layer 107.

One of the most biocompatible materials is Polytetrafluorethylene (PTFE). PTFE can be expanded along one or more axes to gain greater tensile strength in the direction of the expansion, along one axis, this is referred to as the machine direction, under the proper conditions to make a microporous film. In certain embodiments, ePTFE membrane exterior layers 105 and 107 comprise single-axis expanded PTFE. In other embodiments, ePTFE membrane exterior layers 105 and 107 consist essentially of multi-axis expanded PTFE. After expansion, the PTFE film gains tensile strength and low elongation along the axis of expansion, or in the machine expansion direction.

Further, in certain embodiments, ePTFE membrane exterior layers 105 and 107 comprise multiple-axis expanded polytetrafluorethylene (PTFE). In other embodiments, ePTFE membrane exterior layers 105 and 107 consist essentially of multiple-axis expanded PTFE. The more the number of axes along which the PTFE is expanded, the more tensile strength and low elongation the PTFE film gains in all such directions.

An important issue to be addressed is suture retention strength when suture is used to secure the implanted laminate to various tissues within the body, for example, abdominal wall in the example of abdominal wall hernia repair. The suture retention is the ability of the laminate to resist tearing, shearing, pulling, or other damage caused by securing the laminate with sutures. Sutures can shear, tear, or pull through certain materials. Having a laminate that retains the sutures, increases the success of any operation involving a laminate. The present laminate comprises middle layers with various layered, woven, braided, and knitted patterns to increase the suture retention strength, which is measured during a pulling process up to when breakage is determined in accordance with the provisions of the American National Standards Institute/Association for the Advancement of Medical Instrumentation (ANSI/AAMI). Thus, the present laminate is designed to exhibit resistance to tearing, shearing, breaking, etc., that is at least in conformance with national and/or international requirements for implantable devices.

Referring to FIG. 1A layers 101, 102, and 103 comprise tear resistant layers. These layers are made up of ePTFE threads and are similar to the sutures used for attaching the implanted laminate to the tissue it is implanted on. The ePTFE threads strengthen the laminate so as to resist the sutures from tearing, shearing, breaking, or otherwise damaging an implantable laminate. The threads are positioned so that when securing the laminate with sutures the suture will pass through or over the ePTFE threads and not merely suture through layers of ePTFE sheet.

The implantable laminates are constructed to achieve specific qualities. Part of the construction and the qualities comes from the ePTFE threads. Therefore, the ePTFE threads are designed with specific qualities for the implantable laminate. Changing the density of the threads changes the width, porosity, breaking strength, elongation, lamination adherence, and shear strength of the threads. In various embodiments, the density of the threads can be between 0.1 g/cc and 0.9 g/cc, and more specifically between 0.3 g/cc and 0.7 g/cc. For example, the density of the threads can be 0.5 g/cc. More generally, the ePTFE density of the threads is selected according to application-specific needs to support the sheet performance (i.e. flexibility, suture retention, weave design, etc.). The threads in the implantable laminate are designed to interact with the sutures used to secure the laminate to the tissue. The ePTFE threads in the laminate add strength to the laminate so that the securing sutures will not tear through the laminate, or pull the threads through the laminate. In most embodiments, the threads are expanded PTFE. In some embodiments the threads are non-expanded PTFE. Generally, in embodiments utilizing non-expanded PTFE the threads are spaced farther apart so that the laminate maintains some of its porosity.

Combining the layers creates an implantable material with the properties necessary to successfully implant the material into the body. For best success a lamination structure that corresponds to a specific area of the body should be used. These specific lamination structures will be different and will ensure greatest success. For example, a laminate used in conjunction with the pericardium should not allow any tissue ingrowth into the laminate, however a laminate used in conjunction with an artery should allow tissue to grow into the laminate which will further secure the laminate and strengthen the artery, and a laminate used in correcting a hernia should allow tissue ingrowth on one side of the laminate but not on the other. In some embodiments, the exterior layers are laminated to the tear resistant thread layers, by an amorophic porous bonding layer. In other embodiments, the exterior layers are laminated directly to the thread layers.

Referring again to FIG. 1A. Building up the laminate from the bottom up: exterior layer 105 is an ePTFE membrane. Exterior layer 105 may be constructed from single axis ePTFE or from biaxial expanded ePTFE. The preferred thickness of the ePTFE membrane is between 0.1 mm and 0.5 mm. The more preferred thickness of the ePTFE membrane is between 0.2 mm and 0.4 mm. The most preferred thickness of the ePTFE membrane is 0.3 mm. An amorphic ePTFE layer with high porosity is used to connect the exterior layer 105 with the thread layers. In some embodiments, the exterior layers are laminated to the layered thread layers, by an amorophic porous bonding layer. In other embodiments, the exterior layers are laminated directly to the thread layers. Each of the thread layers are laid out in a plane with the threads of each layer running parallel to each other. The threads are spaced apart so that they do not touch the adjacent threads. The distance between the threads ranges from 0.05 mm to 2.5 mm. Each thread is between 0.005 mm and 0.020 mm. In certain embodiments, threads of multiple diameters are used. Multiple thread diameters create a grid pattern with reinforced sections. Thread layer 102 is laid out in a 90° orientation, the threads are spaced so that each thread is not touching any other laid in the same orientation. Thread layer 101 is laid out in a 0° orientation, the threads are spaced so that each thread is not touching any other laid in the same orientation. Thread layer 103 is laid out in a 450 orientation, the threads are spaced so that each thread is not touching any other laid in the same orientation. A second amorphic layer 106 helps bond the second exterior layer 107 to the laminate. Exterior layer 107 may be single axis ePTFE or biaxial ePTFE. In embodiments where the exterior layer 107 is single axis ePTFE, the layer will be oriented so that the axis of expansion is perpendicular or 90° to the axis of expansion of exterior layer 105. By orienting the axis of expansion of exterior layer 105 perpendicular to the axis of expansion of exterior layer 107 the laminate gains tensile strength and limits elongation along both axis. While the invention is described with three tear resistant layers, any number of tear resistant layers can be used. In some embodiments there is a single tear resistant layer of ePTFE threads. In other embodiments there are two tear resistant layers of ePTFE threads.

Once each of the layers have been laid down, the laminate is heated and compressed. The heating and compression bonds or laminates the layers together. The threads are laid down as rods or cylindrical strings. During the lamination process, in some embodiments, the threads are compressed, or flattened. The threads become wider in the horizontal plane, in line with the rest of the threads, and narrower in the vertical plane, perpendicular to the rest of the threads. The flattening of the ePTFE threads in the lamination process, increases the surface area of the threads, which increases the lamination adherence of the threads to the other layers. Lamination adherence or grip strength is the ability of the threads to remain anchored in the laminate and to not pull out of the laminate. In certain embodiments, during the lamination process the laminate in not compressed as tightly and the threads are not flattened.

The exterior layers are the determining layers as to the properties of the laminate, and determine with what tissues the laminate should be used. In certain embodiments the ePTFE membrane exterior layers 105 and 107 are designed so that they will form a barrier to the tissues that the laminate is implanted adjacent to or within. This barrier will not allow tissue to grow into the laminate. Examples of tissues where this is desirable are the pericardium which surrounds the heart. The exterior layers 105 and 107 of the laminate in this embodiment will comprise high density/low porosity ePTFE membranes typically with a porosity of 10-15 microns.

In other embodiments such as artery repair the outer layers 105 and 107 will comprise medium density/medium porosity ePTFE membranes. Typically these will have a porosity of 20-40 microns. This will allow in-growth into the laminate. In some embodiments even greater integration of the laminate to the tissue is desired and low density/high porosity ePTFE membranes will be used.

In yet other embodiments it is desirable that one side of the laminate allow tissue ingrowth while the other side of the laminate acts as a barrier and does not permit any tissue ingrowth. These embodiments would have exterior layer 105 comprise high density/low porosity ePTFE membrane and exterior layer 107 comprise medium density/medium porosity ePTFE membranes.

In certain embodiments, ePTFE membrane exterior 110 and exterior 130 have different porosities from each other and one has a low porosity and the other one has a high porosity. The nodes and fibrils form pores, through which biological tissues, such as muscle fibers, blood vessels, etc., can grow. Tissue growth and attachment to an expanded PTFE film facilitates anchoring and securing the film to the tissue, which is important for its application in medial repair and organ support surgeries. For example, the rate of hernia formation is high among patients who are obese, immunosuppressed, or have had previous abdominal surgery, results in over 2 million laparoscopic hernia repairs every year in the United States. To prevent hernia recurrence, the implanted prosthetic material must be affixed to the abdominal wall (abdominal wall hernias are the most common hernias, therefore, the abdominal wall hernia is used as an example here) and must be able to withstand the pressures generated by coughing, straining, and normal postoperative activity until adequate tissue ingrowth occurs. To ensure incorporation of the implanted material with the abdominal wall, transfascial suture is typically employed.

Referring to FIG. 1B, a clamping rack 150 comprises clamping frames for each layer of the laminate. The layers of the laminate extend past the frame interior. The first exterior layer of ePTFE membrane is placed on base rack 159. The thread layer or layers are placed on thread frame 157. The pegs within the thread frame 157 such as peg 155 are to hold the threads in place. The ePTFE threads are wound around the pegs, such as peg 155. A second exterior layer is placed on frame 153. Upper frame 151 is placed on top of the second exterior layer. The frame pieces are clamped or screwed together. Once the frame is assembled the heating and compression plates are fit within the interior edge of the frame. The laminate is compressed and heated and the layers are bonded together. In some embodiments, amorphic ePTFE is placed between the thread layer and the first and second exterior layer to increase the lamination adherence of the layers.

Once the layers have been arranged on frame 150, the frame 150 is placed within a lamination press such as lamination press of FIG. 1C. The lamination press includes upper hydraulic arm 161, upper lamination plate 162, and lower lamination plate 163. Typically, a lower hydraulic arm will also be a part of the lamination press (in this figure the lower hydraulic arm is obscured by the frame of the lamination press). The frame is secured to the lamination press. Then the upper hydraulic arm 161 and presses the upper lamination plate 162 down onto the laminate and the lower hydraulic arm presses the lower lamination plate 163 up onto the layers as the laminate is held in the frame, the lamination plates are heated by heating elements within the plate, and the layers of the laminate are affixed together creating a single laminate material. In some embodiments, the upper hydraulic arm 161 pushes the lamination plate until it touches the upper outer layer of the laminate, and the lower hydraulic arm pushes the lower lamination plate until it reaches the lower outer layer of the laminate. These embodiments, heat the layers and bond them with heat, the thickness of the laminate in these embodiments is essentially the combined thickness of all the layers. In other embodiments, the upper hydraulic arm 161 pushes the upper lamination plate 162 down and the lower hydraulic arm pushes the lower lamination plate 163 up compressing the layers of the laminate together, while the lamination plates are heated. This binds the layers together with pressure and heat. In addition to binding the layers together with pressure and heat, embodiments that compress the layers together change the overall thickness of the laminate, the thickness of the laminate is reduced as the upper lamination plate 162 and lower lamination plate 163 compress the laminate together. The thickness of the laminate can be specified by a combination of layers and how much pressure is used during the lamination process.

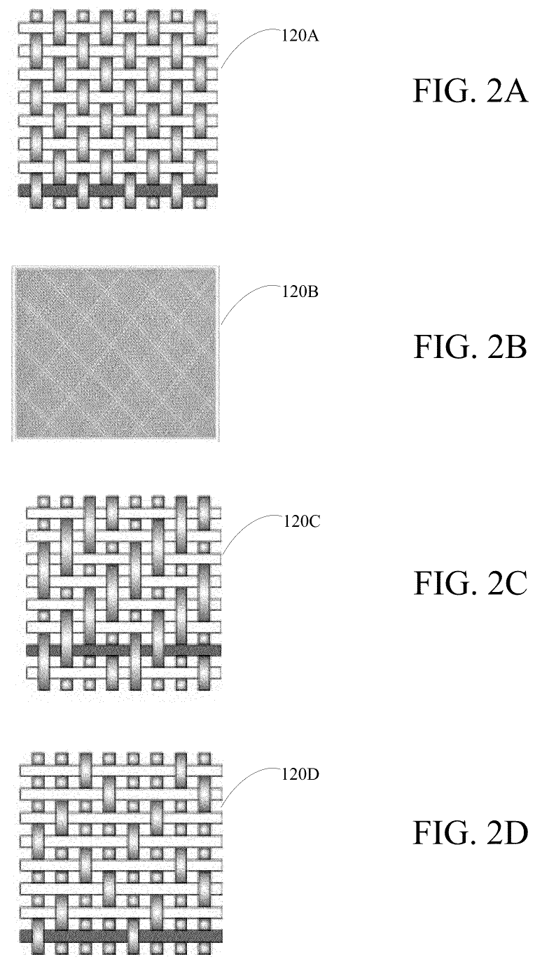

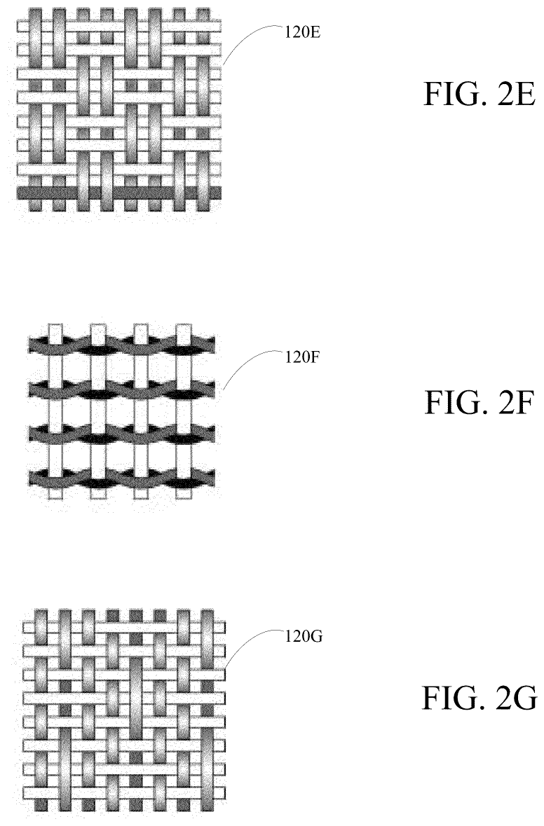

For example and as shown in FIG. 2A, each longitudinal porous PTFE rod passes alternately under and over each lateral porous PTFE rod to produce a symmetrical middle layer 120A with good stability and reasonable porosity. Referring to FIG. 2B, in another example middle layer 120B the longitudinal porous PTFE rods and the lateral porous PTFE rods can be woven into a crosshatch pattern. In another example shown in FIG. 2C, one or more longitudinal porous PTFE rods alternately weave over and under two or more lateral porous PTFE rods to produce a middle layer 120C.

Referring to FIG. 2D, the longitudinal porous PTFE rods and the lateral porous PTFE rods of the middle layer 120D can be woven into a satin weave pattern to produce fewer intersections of warp and weft. Any number of PTFE rods (but typically 4, 5 and/or 8) may be crossed and passed under or over in either direction (i.e., lateral or longitudinal), before the thread repeats the pattern. Referring to FIG. 2E, the longitudinal porous PTFE rods and the lateral porous PTFE rods of the middle layer 120E can be woven into a basket weave pattern, in which two or more warp threads alternately interlace with two or more weft threads.

Referring to FIG. 2F, the longitudinal porous PTFE rods and the lateral porous PTFE rods can be woven into a leno weave pattern to form a middle layer 120F. Leno weave is a weave in which adjacent warp threads are twisted around consecutive weft threads to form a spiral pair, effectively ‘locking’ each weft in place. The leno weave may be used in conjunction with other weave patterns. Additionally or alternatively, as shown in FIG. 2G, the longitudinal porous PTFE rods and the lateral porous PTFE rods of the middle layer 120G can be woven into a mock leno weave pattern, in which occasional warp threads, at regular intervals but usually several threads apart, deviate from the alternate under-over interlacing and instead interlace every two or more threads. This happens with similar frequency in the weft direction.

Further, a thicker reinforcement thread, such as a porous PTFE rod with a greater diameter than 0.002 inches and higher tensile strength, can be interwoven at regular intervals in any of the patterns described above.

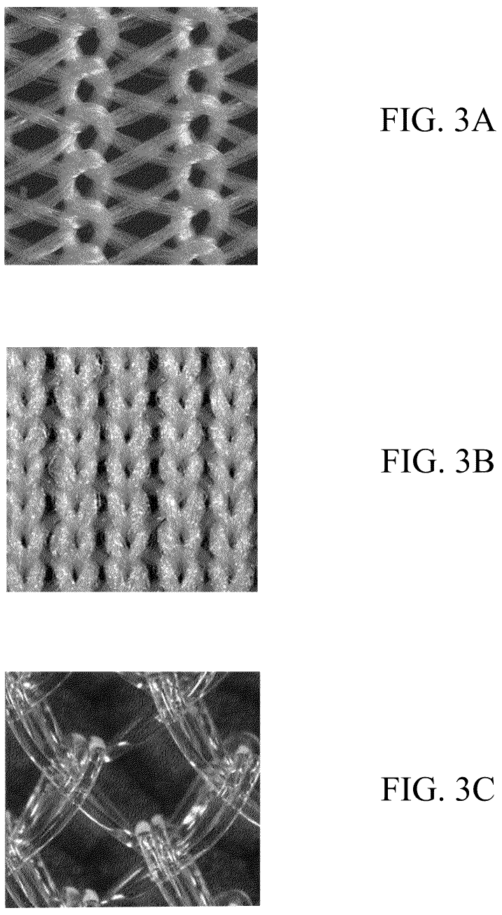

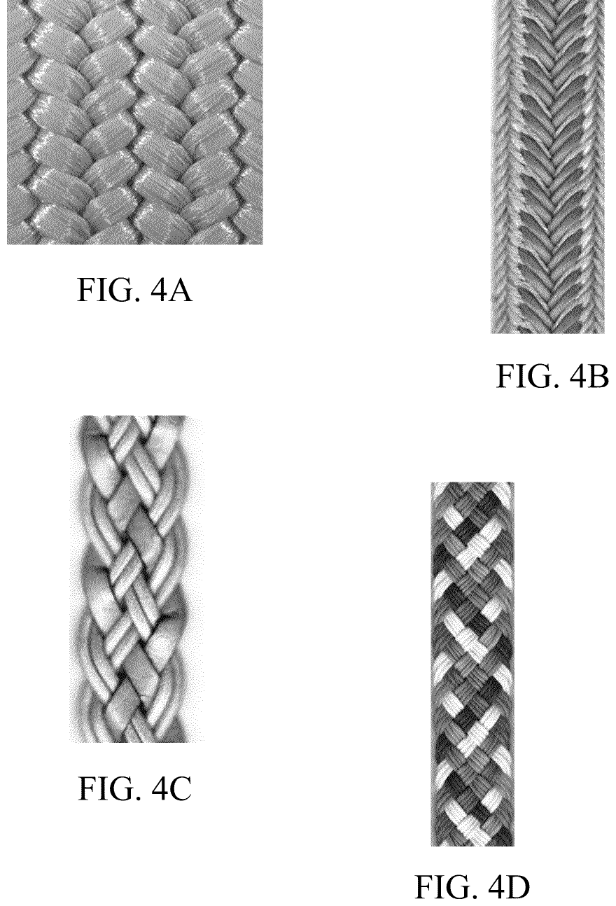

Referring to FIGS. 3A to 3C, additionally or alternatively to weaving, the porous PTFE rods can be knitted with different patterns to form the middle layer with different porosity, tensile strength, and suture retention strength. Similarly, referring to FIGS. 4A to 4D, porous PTFE rods can additionally or alternatively be braided with different patterns to form the middle layer with different porosity, tensile strength, and suture retention strength. Additionally, in certain embodiments, porous PTFE rods can be woven, knitted, braided, or in any combination thereof with different patterns to form the middle layer with different porosity, tensile strength, and suture retention strength.

In certain embodiments a knitted or braided layer is inserted around the periphery of the laminate. The knitted or braided layer does not cover the whole layer, rather it is placed so that the reinforced area is around the periphery of the laminate. This gives added strength to the laminate in places where the laminate is likely to be sutured to the tissue and leaves the majority of the laminate to be comprised of ePTFE membrane.

Each pattern based on the above description is further tested for its suture retention strength. For example, with each middle layer prepared in accordance with a particular pattern, a pinhole is made, a suture is looped through the pinhole, and the suture is attached to a tensile testing machine to study and generate a stress-strain curve for each pattern. The suture retention strength is defined as the peak strength during this testing process.

While the preferred embodiments of the present invention have been illustrated in detail, it should be apparent that modifications and adaptations to those embodiments may occur to one skilled in the art without departing from the scope of the present invention.

We claim:

1. A composite laminate comprising:
a first exterior layer comprising a biocompatible material;
a second exterior layer comprising a biocompatible material; and
a first inner layer comprising fibers running parallel to each other and oriented at zero degrees;
wherein the layers are laminated together.

2. The invention of claim 1, wherein the biocompatible material is ePTFE.

3. The invention of claim 2, wherein the fibers comprise ePTFE.

4. The invention of claim 3, further comprising a second inner layer with the fibers running parallel to one another and oriented at ninety degrees to the first inner layer of fibers.

5. The invention of claim 4, further comprising a third inner layer with the fibers running parallel to one another and oriented at forty-five degrees to the first inner layer of fibers.

6. The invention of claim 5, further comprising a fourth inner layer with the fibers running parallel to one another and oriented at forty-five degrees to the second inner layer of fibers.

7. The invention of claim 5, wherein the first exterior layer and the second exterior layer have an internodal distance between 0.010 mm and 0.020 mm.

8. The invention of claim 5, wherein the first exterior layer and the second exterior layer have an internodal distance between 0.020 mm and 0.080 mm.

9. The invention of claim 5, wherein the first exterior layer has an internodal distance between 0.010 mm and 0.020 mm and the second exterior layer has an internodal distance between 0.020 mm and 0.080 mm.

10. The invention of claim 5, wherein a first amorphic layer is added between the first exterior layer and the inner layers, and a second amorphic layer is added between the inner layers and the second exterior layer.

11. The invention of claim 5, wherein the first exterior layer comprises single axis ePTFE and the second exterior layer comprises single axis ePTFE, wherein the first exterior layer and the second exterior layer are oriented so that their machine expansion axes are perpendicular to one another.

12. The invention of claim 5, wherein the first exterior layer and the second exterior layer comprise biaxial ePTFE.

13. A method for creating a biocompatible composite laminate comprising:
Laying biocompatible fibers parallel to one another to create a first middle fiber layer on a first biocompatible material exterior layer, and placing a second biocompatible exterior material layer over the parallel biocompatible fibers;
heating and compressing the layers to bond the layers together.

14. The invention of claim 1, wherein the biocompatible material is ePTFE.

15. The invention of claim 2, wherein the fibers comprise ePTFE.

16. The invention of claim 3, wherein a second inner layer with the fibers running parallel to one another and oriented at ninety degrees to the first inner layer of fibers is added.

17. The invention of claim 4, wherein a third inner layer with the fibers running parallel to one another and oriented at forty-five degrees to the first inner layer of fibers is added.

18. The invention of claim 5, wherein the first exterior layer and the second exterior layer have an internodal distance between 0.010 mm and 0.020 mm.

19. The invention of claim 5, wherein the first exterior layer has an internodal distance between 0.010 mm and 0.020 mm and the second exterior layer has an internodal distance between 0.020 mm and 0.080 mm.

20. The invention of claim 5, wherein a first amorphic layer is added between the first exterior layer and the inner layers, and a second amorphic layer is added between the inner layers and the second exterior layer.
